(19) United States
(12) Patent Application Publication
(10) Pub. No.: US 2016/0367370 A1
(43) Pub. Date: Dec. 22, 2016

(54) ORTHOPEDIC SURGICAL INSTRUMENT SET

(71) Applicant: Corentec Co., Ltd., Cheonan-si (KR)

(72) Inventors: Seok-Joo Kim, Gwangju-si (KR); Oui-Sik Yoo, Seoul (KR); Michael Santarella, Essex Fells, NJ (US)

(73) Assignee: Corentec Co., Ltd.

(21) Appl. No.: 15/188,459

(22) Filed: Jun. 21, 2016

Related U.S. Application Data
(60) Provisional application No. 62/183,076, filed on Jun. 22, 2015.

(51) Int. Cl. A61F 2/32; A61F 2/40; A61F 2/00; A61F 2/38

(52) U.S. Cl. CPC A61F 2/32; A61F 2/38; A61F 2/40; A61F 2/0095; A61F 2002/30616

(57) ABSTRACT

The present invention relates to an orthopedic surgical set and, more particularly, to an orthopedic surgical set in which an implant having a specific size, which is determined and selected in advance on the basis of scanning information obtained through a medical scanner, to be implanted into a human body through an operation, spare implants, which have a size selected within an error range in preparation for error that may occur between the scanning information and the actual information of a patient, and different kinds of surgical instruments, which correspond to the sizes of the implants, are classified by sizes and fixed in k-th trays, respectively, to be kept in a container so that a doctor who performs an operation can easily select desired surgical instruments, costs for sterilizing the surgical instruments are decreased, and the tray and the container can be reduced in size for easy carrying.

### CROSS-REFERENCE TO RELATED APPLICATIONS

This application claim priority to US Provisional Application No. 62/183,076, filed Jun. 22, 2015, which is incorporated herein by specific reference.

### BACKGROUND OF THE INVENTION

1. Field of the Invention

The present invention relates to an orthopedic surgical set and, more particularly, to an orthopedic surgical set in which an implant having a specific size, which is determined and selected in advance on the basis of scanning information obtained through a medical scanner, to be implanted into a human body through an operation, spare implants, which have a size selected within an error range in preparation for errors that may occur between the scanning information and the actual information of a patient; and different kinds of surgical instruments, which correspond to the sizes of the implants, are classified by sizes and fixed in k-th trays, respectively, to be kept in a container so that a doctor who performs an operation can easily select desired surgical instruments, costs for sterilizing the surgical instruments are decreased, and the tray and the container can be reduced in size for easy carrying.

2. Description of the Related Art

For implant surgery, various surgical instruments are required to cut a portion of a bone, place an implant, and fix the bone, and the surgical instruments are fixed and sterilized on a tray or a container.

FIG. 1 is a perspective view showing a tray system with surgical instruments fixed in trays.

Referring to FIG. 1, in the related art, surgical instruments S of all sizes for an implant surgery on a specific portion are all fixed on a tray T and then kept in a container, and they are sterilized therein. For example, when rasps are used as surgical instruments for an operation and are classified into a 1-Ho to 12-Ho in accordance with the sizes, generally, not only the rasps of specific sizes for the operation, but the other rasps are all fixed on a tray T and then kept in a container, and they are all simultaneously sterilized therein.

When surgical instruments S are kept on the medical tray T, not only the surgical instruments S having sizes necessary for implant surgery, but surgical instruments S having other sizes not necessary for the operation are kept on the tray T, so the medical tray T has to be large for sterilization, thus increasing the cost of the tray T.

Further, according to the medical tray T, as described above, since surgical instruments S having sizes that are not used in an operation are kept in a container in the related art, a doctor cannot quickly select surgical instruments S having sizes suitable for implants of specific sizes during an operation, so the operation time is increased and the patient may surfer from aftereffects due to excessive bleeding.

Further, since the surgical instruments S having sizes that are little used are also kept and sterilized on the tray T, the cost for sterilizing is increased, which consequently increases the cost for an operation, so the patient has to bear more expenses.

As another problem, since surgical instruments having sizes that are not frequently used are also necessarily kept on the medical trays, it is difficult to freely arrange the surgical instruments and it is difficult to carry the containers due to an increase in size of the containers. Further, the prices of the containers are also increased.

Korean Patent No. 10-1478314, titled “Tray for medical use”, disclosed a tray that includes a kit keeping surgical instruments and a cover being in contact with each other by a closing unit to prevent damage due to mixing of the surgical instruments when the tray is carried and that is convenient to use without rearranging the surgical instruments, thereby preventing delay of an operation.

This document also did not propose the object of fixing surgical instruments having sizes that have a probability to be used in an operation in a medial tray, so the problems such as an increase in costs of a tray and a container, an increase in cost of an operation due to an increase in cost for sterilization, and difficulty in carrying the container are likely to be generated.

### SUMMARY OF THE INVENTION

The present invention has been made in an effort to solve the problems.

An object of the present invention is to provide an orthopedic surgical set that can keep only different kinds of surgical instruments having sizes corresponding to a specific implant, which is determined on the basis of scanning information obtained by a medical scanner, in one tray together with the implant so that a doctor performing an operation can quickly and easily select the desired surgical instruments having sizes for the operation, thereby preventing delay of the operation.

Another object of the present invention is to provide an orthopedic surgical set that can keep different kinds of surgical instruments having sizes corresponding to spare implants, which are selected within an error range in consideration of errors that may occur between scanning information and the actual information of a patient in order to perform an implant surgery, in additional trays together with the spare implant so that a doctor performing the operation can quickly and easily select the desired surgical instruments having sizes for the operation, and that can reduce costs by reducing the size of a container for the tray.

Another object of the present invention is to provide an orthopedic surgical set that can reduce the size of a container so that the container can be easily carried, by classifying an implant based on scanning information, spare implants, and only different kinds of surgical instruments having sizes corresponding to the implants by the sizes and then by fixing them in separate trays so that a container can be reduced in size to be easily carried.

Another object of the present invention is to provide an orthopedic surgical set that can simplify operation, reduce costs, and improve convenience for carrying a container by reducing the size of the container because it has one to three tray units, preferably, three tray units.

Another object of the present invention is to provide an orthopedic surgical set that can achieve practicability and flexibility of a configuration by keeping implants that are larger and smaller by one size than an implant based on scanning information as spare implants in consideration of errors between the scanning information and the actual information.

Another object of the present invention is to provide an orthopedic surgical set that can reduce costs for sterilizing using high-temperature vapor blown through holes of a tray by keeping only surgical instruments having sizes for an implant surgery on the tray together with a plurality of implants.

Another object of the present invention is to provide an orthopedic surgical set that allows a user to purchase a tray and a container at low costs by providing a container that keeps a tray for keeping only surgical instruments having sizes that are frequently used in implant surgery.

According to an embodiment of the present invention, an orthopedic surgical set includes: k-th set units where implants having different sizes selected on the basis of scanning information of a patient obtained by a medical scanner and selected in consideration of a range of errors, which may occur between the scanning information and actual information, to perform implant surgery on a specific portion, and different kinds of surgical instruments having sizes corresponding to the implants to be used in the implant surgery are classified by sizes together with corresponding implants; and k-th tray units to which the k-th set units are fixed, respectively.

The k-th set units may each include: an implant unit including one scanning information implant selected on the basis of the scanning information and one or more spare implants selected within the range of errors that may occur between the scanning information and the actual information; and n-k-th surgical instruments used to perform the implant surgery and obtained by classifying different kinds of surgical instruments having sizes corresponding to the scanning implant and the spare implants by sizes corresponding to the implants, respectively.

The number of the k-tray unit may be the same of the number of the implants included in the implant unit.

The number of the spare implants may be one to three.

The number of the spare implants may be two.

The two spare implants may be spare implants larger and smaller by one size than the scanning information implant.

Surgical instruments having one size or different kinds of implants may be fixed in a spare space of any one of the k-trays.

The implant unit may include any one kind of implant of implants that are used for artificial knee implant surgery.

The implant unit may include any one kind of implant of implants that are used for artificial hip joint implant surgery.

The implant unit may include any one kind of implant of implants that are used for artificial shoulder implant surgery.

The k-th tray may include: brackets protruding upward in a predetermined shape from a bottom thereof to fix surgical instruments and implants for an operation; and handles formed in a predetermined shape at both side of a top of the k-th tray for a user to easily hold the k-th tray.

The orthopedic surgical may further include a rectangular parallelopiped container having an open top to receive one or more trays therein.

A plurality of holes may be formed through a front side or a predetermined side of the trays and the container so that high-temperature vapor for sterilization flows into the trays and the containers.

The present invention can obtain the following effects from the embodiments, and the configurations and combination and use relationships to be described below.

According to an orthopedic surgical instrument set of the present invention, it is possible to keep only different kinds of surgical instruments having sizes corresponding to a specific implant, which is determined on the basis of scanning information obtained by a medical scanner, in one tray together with the implant so that a doctor performing an operation can quickly and easily select the desired surgical instruments having sizes for the operation, thereby preventing delay of the operation.

Further, it is possible to keep different kinds of surgical instruments having sizes corresponding to spare implants, which are selected within an error range in consideration of errors that may occur between scanning information and the actual information of a patient in order to perform an implant surgery, in additional trays together with the spare implant so that a doctor performing the operation can quickly and easily select the desired surgical instruments having sizes for the operation, and that can reduce costs by reducing the size of a container for the tray.

Further, it is possible to reduce the size of a container so that the container can be easily carried, by classifying an implant based on scanning information, spare implants, and only different kinds of surgical instruments having sizes corresponding to the implants by the sizes and then by fixing them in separate trays so that a container can be reduced in size to be easily carried.

Further, it is possible to simplify operation, reduce costs, and improve convenience for carrying a container by reducing the size of the container because it has one to three tray units, preferably, three tray units.

Further, it is possible to achieve practicability and flexibility of a configuration by keeping implants that are larger and smaller by one size than an implant based on scanning information as spare implants in consideration of errors between the scanning information and the actual information.

Further, it is possible to reduce costs for sterilizing with high-temperature vapor blown through holes of a tray by keeping only surgical instruments having sizes for an implant surgery on the tray together with a plurality of implants.

Further, it is possible to allow a user to purchase a tray and a container at low costs by providing a container that keeps a tray for keeping only surgical instruments having sizes that are frequently used in implant surgery.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a perspective view showing a tray system with surgical instruments fixed on trays;

FIG. 2 is an assembly view of an orthopedic surgical set according to an embodiment of the present invention;

FIG. 3 is an exploded view of an orthopedic surgical set according to an embodiment of the present invention;

FIG. 4 is an exploded perspective view when surgical instruments are fixed on a k-th tray shown in FIG. 3;

FIG. 5 is a block diagram of a k-th tray unit according to an embodiment of the present invention;

FIG. 6 is a perspective view of the k-th tray unit shown in FIG. 5;

FIG. 7 is a perspective view of the k-th tray shown in FIG. 5;

FIG. 8 is a block diagram of the k-th set unit shown in FIG. 5;

FIG. 9 is a block diagram of an implant unit shown in FIG. 8;

FIG. 10 is a block diagram of an n-k-th surgical instrument shown in FIG. 8;

FIG. 11 is an exploded perspective view of a container shown in FIG. 2;

FIG. 12 is a plan view of a k-th tray that is used for femoral implant surgery of knee implant surgery according to an embodiment of the present invention;

FIG. 13 is a plan view showing a k-th tray unit formed by fixing a k-th set unit to a k-th tray shown in FIG. 12; and

FIG. 14 is a plan view when a tibial base plate is additionally fixed to the k-th unit shown in FIG. 13.

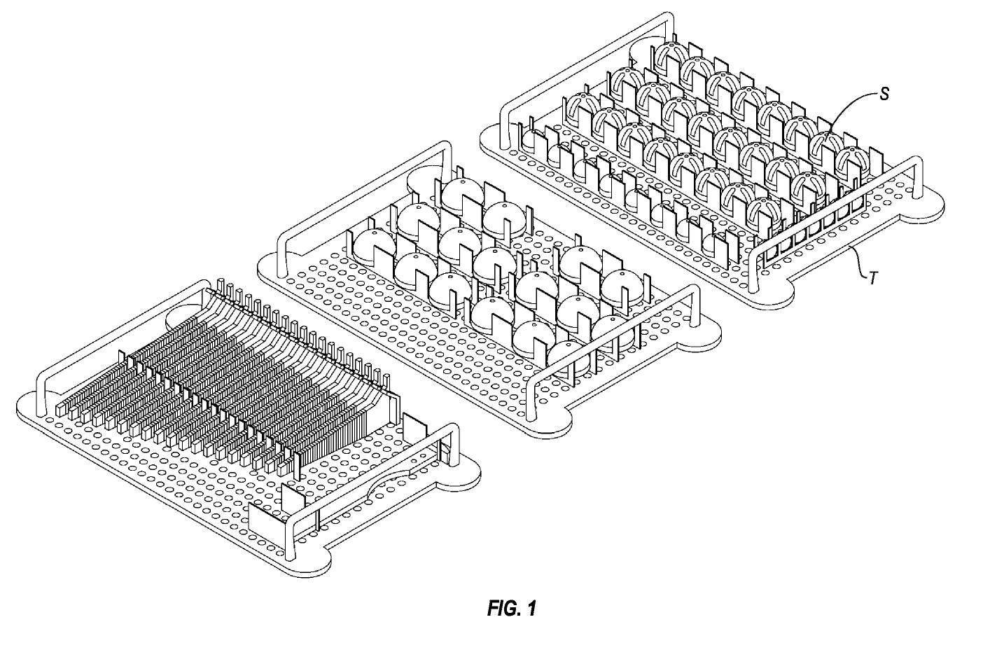

### DETAILED DESCRIPTION OF THE INVENTION

Hereinafter, exemplary embodiments of an orthopedic surgical set according to the present invention will be described with reference to the accompanying drawings. Further, in describing the present invention, well-known functions or constructions will not be described in detail since they may unnecessarily obscure the understanding of the present invention.

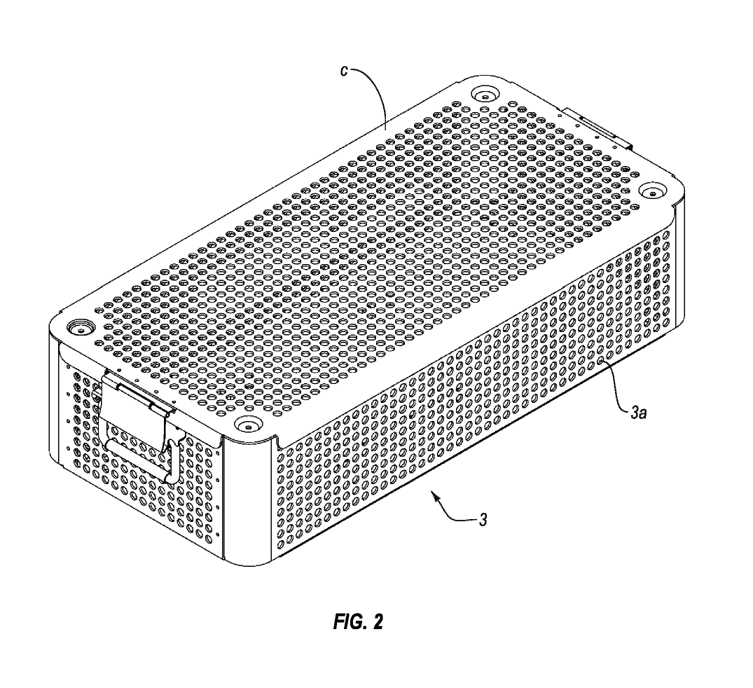

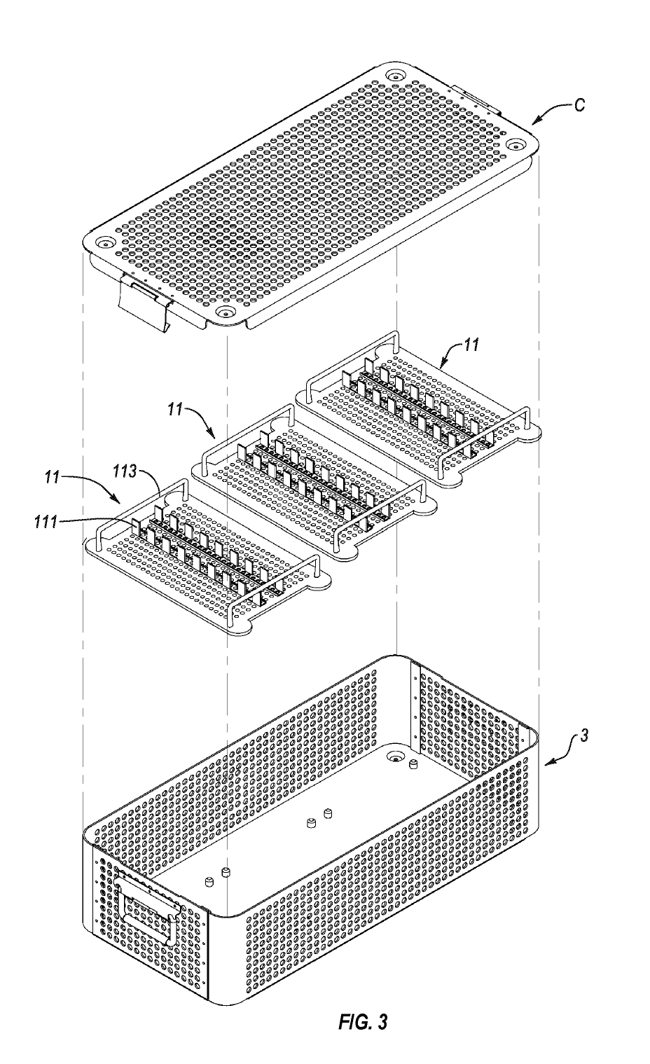

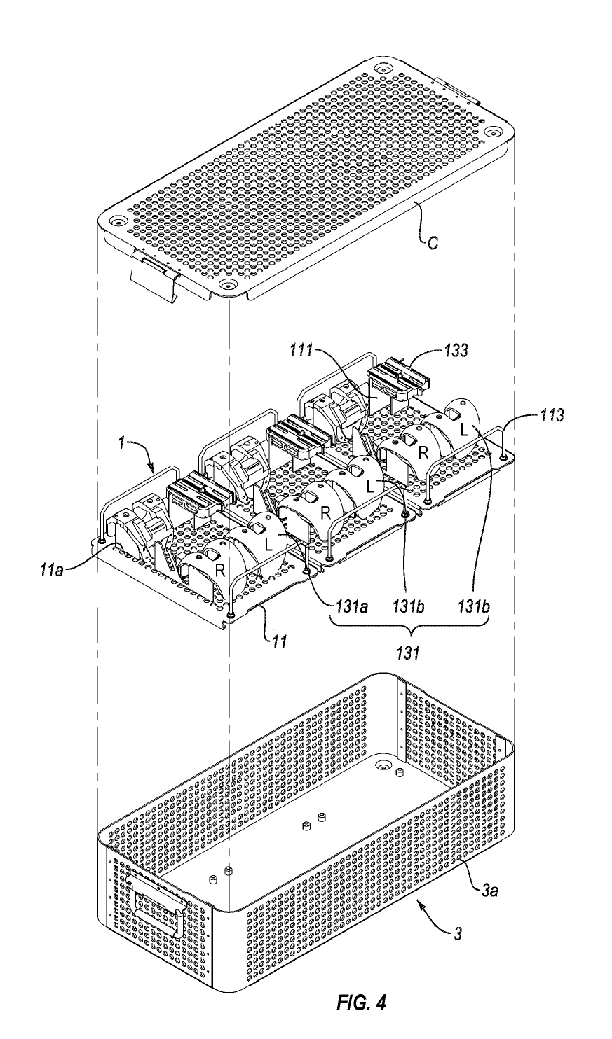

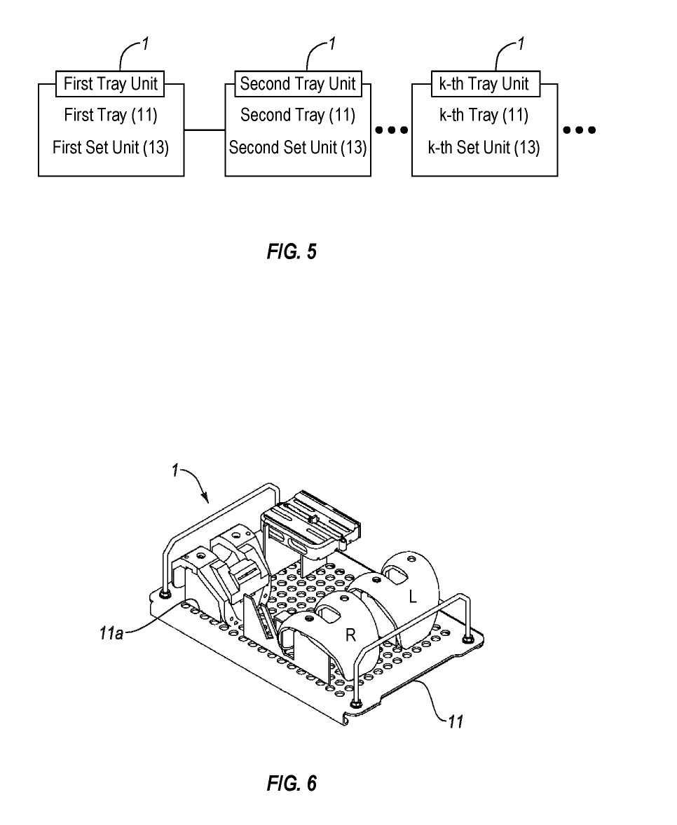

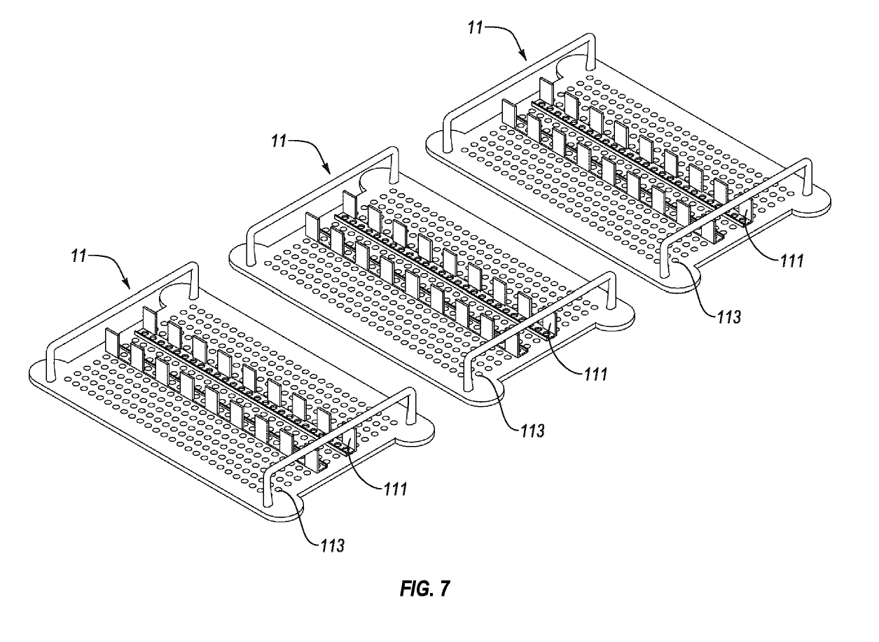

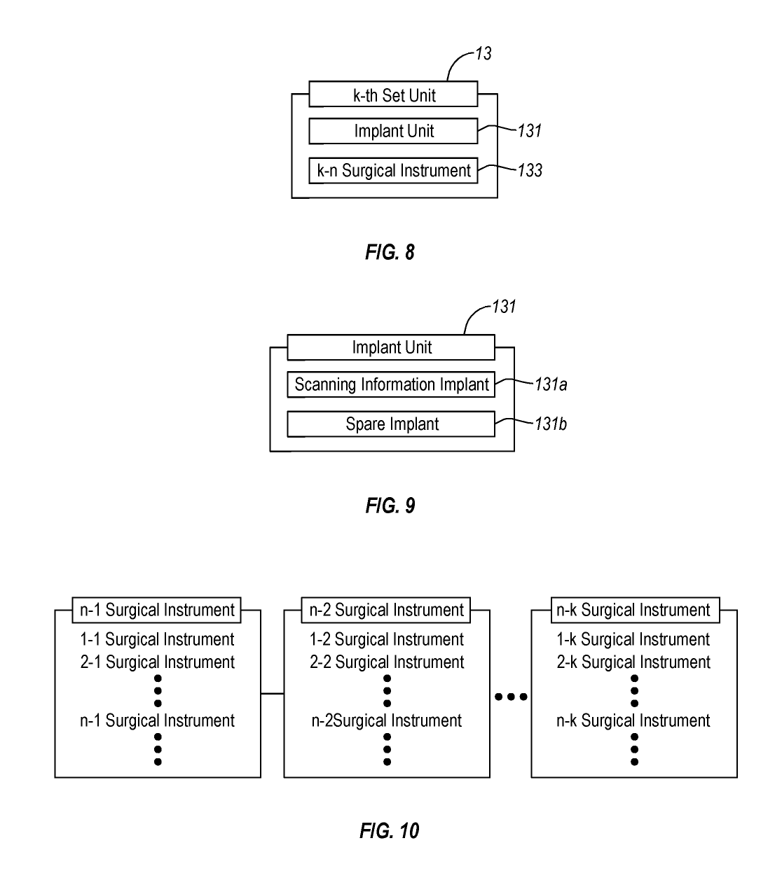

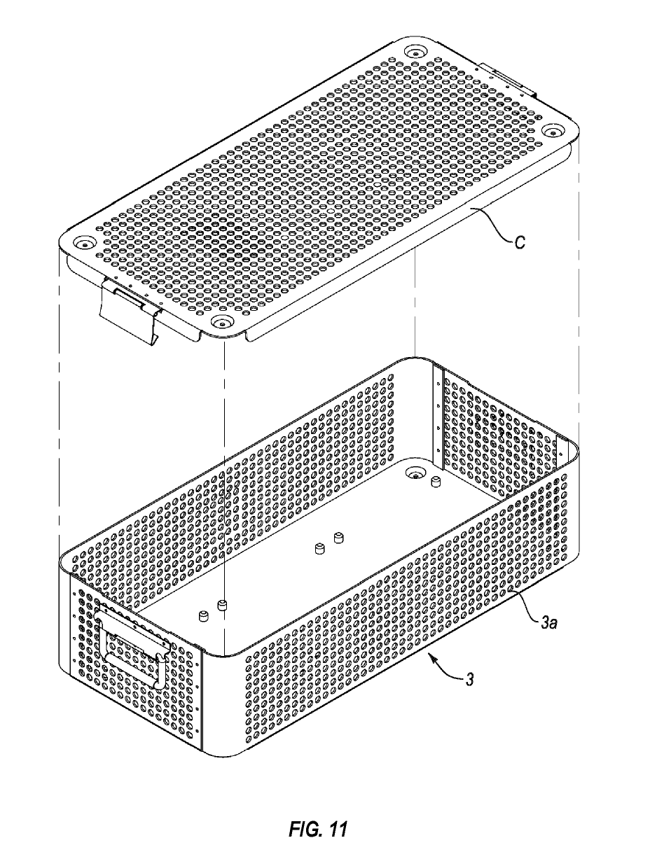

FIG. 2 is an assembly view of an orthopedic surgical set according to an embodiment of the present invention, FIG. 3 is an exploded view of an orthopedic surgical set according to an embodiment of the present invention, FIG. 4 is an exploded perspective view when surgical instruments are fixed on a k-th tray shown in FIG. 3, FIG. 5 is a block diagram of a k-th tray unit according to an embodiment of the present invention, FIG. 6 is a perspective view of the k-th tray unit shown in FIG. 5, FIG. 7 is a perspective view of the k-th tray shown in FIG. 5, FIG. 8 is a block diagram of the k-th set unit shown in FIG. 5, FIG. 9 is a block diagram of an implant unit shown in FIG. 8, FIG. 10 is a block diagram of an n-k-th surgical instrument shown in FIG. 8, and FIG. 11 is an exploded perspective view of a container shown in FIG. 2.

Referring to FIGS. 2 to 4, an orthopedic surgical set according to an embodiment of the present invention includes: a tray 11 for fixing an implant having a specific size selected on the basis of scanning information of a patient obtained by a medical scanner (or a trial, hereafter, the implant selected on the basis of scanning information are all referred to an implant), and a set of different kinds of surgical instruments having sizes corresponding to the implant of surgical instruments that are used for the implant; and an additional tray 11 for fixing a spare implant having a size selected within an error range from the implant selected on the basis of the scanning information in consideration of errors that may occur between the scanning information and the actual information of a patient, and a set of different kinds of surgical instruments having sizes corresponding to the spare implant. To this end, the orthopedic surgical set may include a k-th tray unit 1 and a container 3. The term ‘medical scanner’ used herein is a certain scanner well known in the art such as an X-ray scanner, CT device, and an MRI device, but is not limited thereto.

Referring to FIGS. 5 and 6, the k-th tray unit 1 is a single unit in which implants, which have different sizes selected on the basis of scanning information of a patient obtained by a medical scanner and selected within a range of errors that may occur between the scanning information and the actual information of the patient, and different kinds of surgical instruments S having sizes corresponding to the implants, respectively, are grouped by sizes and fixed on the trays 11, respectively, (k: an integer of 1 or more). The k-th tray units 1 generally each include one implant of a different size, so the number of the k-th tray units 1 is the same as the number of implants selected for implant surgery. The implants that are included in the k-th tray units 1, respectively, are only the same kinds of implants to be implanted in the same portions of the patient' body. Accordingly, for a knee implant, it is not excluded that not only a femoral component and a tibial base plate are included in the same tray unit 1, but a left knee implant and a right knee implant are included in the same tray unit 1. Further, the ‘surgical instruments’ means all kinds of surgical instruments having a plurality of sizes of surgical instruments that are used to specific implants in an implant surgery.

The k-th tray unit 1 is described in detail. For example, when three implants having different sizes are selected in consideration of scanning information and actual information, three tray units 1 are required to individually fix the implants and different kinds of surgical instruments S having sizes corresponding to the implants in accordance with sizes, which may be called respectively a first tray unit, a second tray unit, and a third tray unit. In general, since errors that may occur between scanning information and actual information of a patient are not large, a total of three implants, that is, an implant having a specific size based on the scanning information and implants that are larger and smaller by one size than the implant based on the scanning information and are selected in consideration of the actual information, may be selected. Accordingly, the surgical set according to the present invention is generally composed of the first to third tray units 1, but the scope of the present invention is not limited thereto. The k-th tray unit 1 may include a k-th tray 11 and a k-th set unit 13.

Referring to FIG. 7, the k-th tray 11 is a part in which an implant(s) selected for implant surgery and different kinds of surgical instruments S having sizes corresponding to the implant(s) are grouped by sizes and fixed, and may have a plate shape. A plurality of holes 11a is formed through the bottom of the k-th tray 11 to sterilize surgical instruments S fixed thereon. Accordingly, the tray 11 may be made of aluminum or rigid materials that can resist high temperature. The k-th tray 11 is described in detail. For example, when three tray units 1, that is, the first tray unit, second tray unit, and third tray unit are used, there are provided three trays 11, that is, a first tray included in the first tray unit, a second tray included in the second tray unit, and a third tray included in the third tray unit. Further, when a plurality of trays 11 is kept in a container 3 to be described below, the trays 11 may be vertically arranged such as a top tray and a bottom tray etc., may be horizontally arranged in the same plane, or may be horizontally arranged in parallel with each other, so the way of arranging the trays 11 is not limited. The k-th trays 11 may each include brackets 111 and handles 113.

The brackets 111 protrude upward in a predetermined shape on the bottom of the k-th trays 11 to fix various surgical instruments and implants for an operation. The number and shape of the brackets 111 on the k-th trays 11 are variable and may depend on the type of implant surgery and the sizes of implants, but they are not limited.

The handles 113 are formed in a predetermined shape at both sides on the top of the k-th tray 11 for a user to easily hold the k-th tray, so the k-tray 11 can be easily put into the container 3. The handles 113 may be formed in a U-shape and arranged in a pair to face each other at both sides of each of the k-th trays 11.

Referring to FIGS. 5 and 8, the k-th set unit 13 is a single unit where a plurality of implants selected on the basis of scanning information obtained by a medical scanner and the actual information of a patient and sets of different kinds of surgical instruments having sizes corresponding to the implants are grouped by sizes. That is, a certain k-th set unit 13 includes an implant having a specific size and a set of different kinds of surgical instruments having sizes corresponding to the implant. Accordingly, an individual set unit 13 is fixed in each k-th tray 11, so the numbers of the set units 13 and the tray units 1 are the same. That is, the first set unit 13 is fixed in the first tray 11, the second set unit 13 is fixed in the second tray 11, and the third set unit 13 is fixed in the third tray 11, thereby forming the first to third tray units 1. Further, for example, surgical instruments having a single size regardless of the size of the implant may be fixed in a specific tray 11, or in a spare space of a certain k-th tray 11. The k-th set unit 13 may include an implant unit 131 and an n-k surgical instrument 133.

Referring to FIGS. 4 and 9, the implant unit 131 is composed of an implant selected on the basis of scanning information of a patient and an implant selected within a range of errors that may occur between the scanning information and the actual information. The configuration of the implant unit 131 may be change in accordance with the types of implants to be implanted into the body of a patient. That is, the implant unit includes implants having specific sizes that are selected on the basis of scanning information and actual information and are variable in accordance with the types of surgeries such as artificial keen implant surgery, artificial hip joint implant surgery, and artificial shoulder joint implant surgery. To this end, the implant unit 131 may include a scanning information implant 131a and a spare implant 131b.

The scanning information implant 131a is an implant having a specific size selected on the basis of scanning information of a patient, in detail, is one implant variable in accordance with the portion of a patient to be operated. For example, the scanning implant may be a femoral component or a tibial component in artificial knee joint surgery, an actabular cup in artificial hip joint implant surgery, and a glenoid implant or a humerus implant in artificial shoulder joint implant surgery, but this is proposed only for describing the present invention and the present invention is not limited thereto.

The spare implant 131b, which is an addition implant(s) selected within a range of errors that may occur between the scanning information and the actual information of a patient, depends on the portion of a patient to be operated, similar to the scanning information implant 131a. One to three implants may be provided for the spare implant 131, and as described above, since the errors that may occur between the scanning information and the actual information of a patient are usually not large, a total of two implants, that is, implants larger and smaller by one size than the scanning information implant 131a selected on the basis of the scanning information may be selected.

Referring to FIG. 10, the n-k surgical instrument 133 includes different kinds of surgical instruments that have sizes corresponding to implants included in the implant unit 131 and are grouped by sizes corresponding to the implants to perform a specific implant surgery (n: integer of 1 or more). Accordingly, the n-k surgical instrument 133 is also variable in accordance with the types of implants. First, a set of a same kind of surgical instruments having all sizes of surgical instruments that are used for an operation is defined as an n-th surgical unit (n: integer or 1 or more). For example, when rasps are used as surgical instruments, the rasps are classified into a 1-Ho to a 12-Ho in accordance with the sizes, and in this case, the set of the rasps may be called a first surgical unit, and the 1-Ho rasp is called a 1-1 surgical instrument, the 2-Ho rasp may be called a 1-2 surgical instrument, and the 12-Ho rasp may be called a 1-12 surgical instrument. A set of another kind of surgical instruments other than the raps may be called a first surgical unit and the set of rasps may be called a second unit, in which the 1-Ho rasp may be called a 1-12 surgical instrument, but there is no limit. However, when the numbers corresponding to k of different kinds of surgical instruments are the same, they correspond to the same size of implant, so they constitute k-th set unit 13 together with the implant, and the k-th set unit 13 is fixed to the k-th tray 11, thereby constituting the k-th tray unit 1. Accordingly, the 1-1 surgical instrument, 2-1 surgical instrument, . . . , and n-1 surgical instrument 133 constitute the first set unit 13, the 2-1 surgical instrument, the 2-2 surgical instrument, . . . , and the n-2 surgical instrument 133 constitute the second set unit 13, the first set unit 13 is fixed to the first tray 11, thereby constituting the first tray unit 1, and the second set unit 13 is fixed to the second tray 11, thereby constituting the second tray unit 1.

Referring to FIGS. 2 and 11, the container 3, which may receive one or more trays 11, may be formed in a rectangular parallelepiped with an open top. The container 3 is high enough for coupling a lid C to the top of the container 3 with trays in the container 3. Further, a plurality of holes 3a is formed through the front side or any one side of the container 3 so that high-temperature vapor for sterilization can flow into the container. Since the surgical instruments in the container 3 need to be sterilized at high temperature, the container 3 may be made of aluminum or a rigid material that can resist high temperature and the volume of the container 3 may be variously changed in accordance with the number and size of the tray units 1 to be kept therein.

A femoral implant surgery of knee implant surgery is exemplified in the following description, but it is just an example and those skilled in the art may understand that embodiments described in the specification and claims can be applied to other implant surgeries in the same way without a limit.

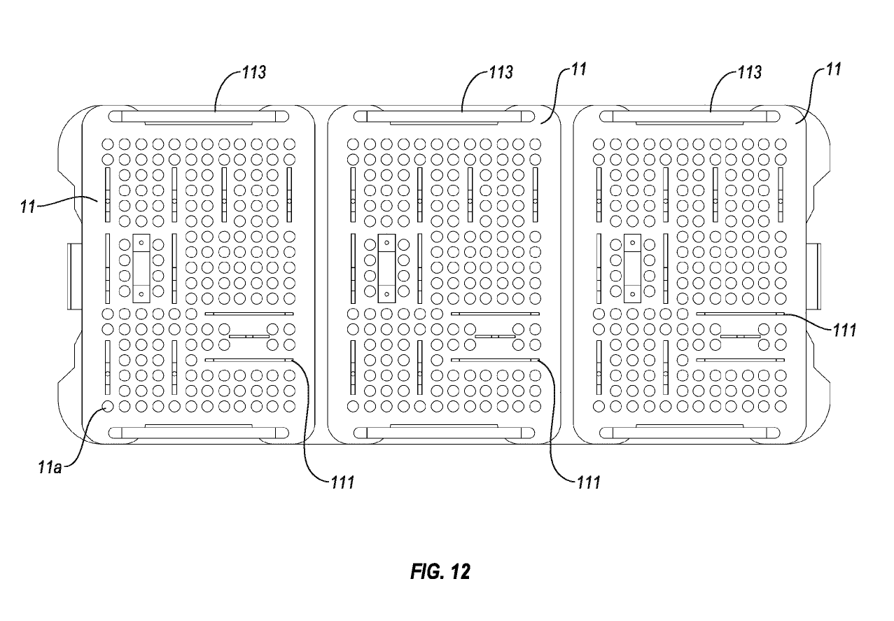

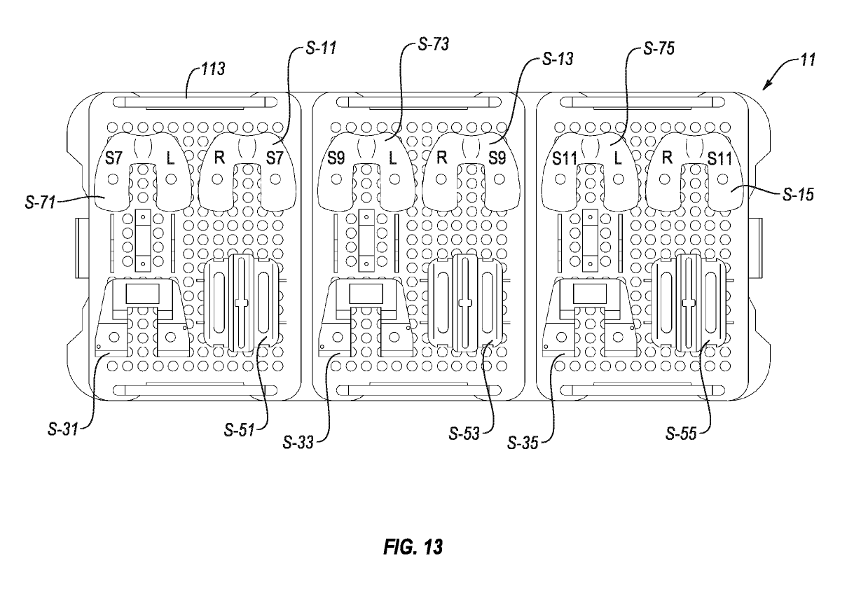

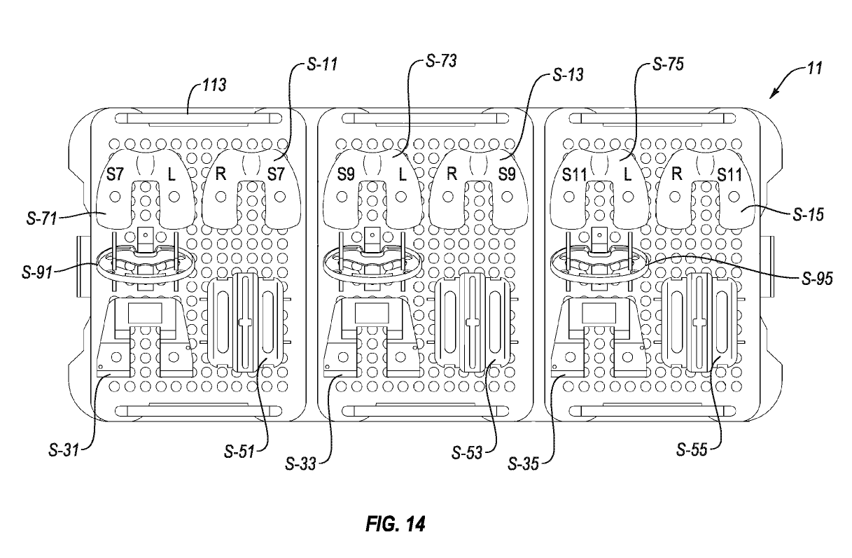

FIG. 12 is a plan view of a k-th tray that is used for femoral implant surgery of knee implant surgery according to an embodiment of the present invention, FIG. 13 is a plan view showing a k-th tray unit formed by fixing a k-th set unit to a k-th tray shown in FIG. 12, and FIG. 14 is a plan view when a tibial base plate is additionally fixed to the k-th unit shown in FIG. 13.

Referring to FIGS. 12 and 13, according to an embodiment of the present invention, when a femoral component implant surgery is performed using Lospa Knee System by Corentec Co. Ltd, right femoral trials 131a, 151a, and 171a, PS box cutting guides 131b, 151b, and 171b, and chamfer cutting guides 131c, 151c, and 171c are used. The set of femoral trials S-51, S-53, and S-55 may be called a first surgical unit, the set of PS box cutting guides S-31, S-33, and S-35 may be called a second surgical unit, and the set of S-51, S-53, and S-55 may be called a third unit. Further, when there are six sizes of the femoral trials, they may be called a 1-1 surgical instrument, a 1-2 surgical instrument, . . . , and a 1-6 surgical instrument; the PS box cutting guides may also be called a 2-1 surgical instrument, a 2-2 surgical instrument, . . . , and a 2-6 surgical instrument in accordance with sizes corresponding to the femoral components; and the chamfer cutting guides may also be called a 3-1 surgical instrument, 3-2 surgical instrument, . . . , and 3-6 surgical instrument. Further, when the size of a femoral component determined to be implanted into the body of a patient on the basis of scanning information obtained by a medical scanner is 9, three femoral trials, that is, 7-sized (131a), 9-sized (151a), and 11-sized (171a) femoral trials corresponding to 7-sized, 9-sized, and 11-sized femoral components may be selected in consideration of errors, and they may be called a 1-1 surgical instrument, a 1-2 surgical instrument, and a 1-3 surgical instrument. Further, three sizes of PS box cutting guides (S-31, S-33, and S-35) corresponding to the femoral components of the selected sizes may be selected and they may be called a 2-1 surgical instrument, a 2-2 surgical instrument, and a 2-3 surgical instrument, respectively. Further, three sizes of chamfer cutting guides (S-51, S-53, and S-55) may be selected and they may be called a 3-1 surgical instrument, a 3-2 surgical instrument, and a 3-3 surgical instrument, respectively.

The selected surgical instruments are fixed to the k-tray 11; different kinds of surgical instruments having sizes corresponding to implants, that is, the first set unit 13 composed of the 1-1 surgical instrument, the 2-1 surgical instrument, and the 3-1 surgical instrument may be fixed to the first tray 11; the second set unit 13 composed of the 1-2 surgical instrument, the 2-2 surgical instrument, and the 3-2 surgical instrument may be fixed to the second tray 11; and the third set unit composed of the other surgical instruments may be fixed to the third tray 11. Accordingly, as the k-th set units are fixed to the k-th trays 11, respectively, the first tray unit, the second tray unit, and the third tray unit are achieved. Further, a twist drill, a guide drill, a distal cutting drill guide and a distal cutting guide tower, and a femoral redriller, which each have one size and are used for implanting a femoral component, may be fixed in the spare spaces of the trays 11, 15, and 17, or specific trays. Further, as described above, the ‘implant’ that is included in each of the k-th tray units 1 means only the same kinds of implants to be implanted in the same portions of a patient, so the left femoral trials S-71, S-73, and S-75 and/or the tibial base plate trials S-91, S-93, and S-95 can be fixed in the spare spaces of the k-th trays 11 together with the right femoral trials S-11, S-13, and S-15 (see FIG. 14).

Further, there are a CR type and a PS type for artificial knee surgery in the related art. The CT type is used for an artificial knee that is inserted without a posterior cruciate ligament removed and the PS type is used for an artificial knee for replacing a posterior cruciate ligament with a bearing with the posterior cruciate ligament removed. Surgical instruments and implants to be fixed to the k-th trays are different in accordance with the PS type or the CR type when an artificial knee is implanted, and a CR type tray where various implants and surgical instruments for CR type surgery are fixed and a PS type tray where various implants and surgical instruments for PS type surgery are fixed can be selectively received in the same container e for flexibility and economic efficiency of an operation. That is, it is possible to insert the CR type tray 11 first in the container 3, and in order to perform PS type surgery, it is possible to separate the CR type tray 11 from the container 3 and then insert the PS type tray instead of the CR type tray, and the opposite case is possible.

Therefore, according to an orthopedic surgical set of the present invention, when the size of an implant to be implanted in the body of a patient is determined on the basis of scanning information obtained by a medial scanner, sets of different kinds of surgical instruments having sizes corresponding to the implant and sets of different kinds of surgical instruments having sizes corresponding to other implants having sizes selected within an error range in consideration of errors that may occur between the scanning information and the actual information are classified by sizes and fixed to the brackets 11 of the k-th trays 11, respectively, together with corresponding implants. Accordingly, the amount of surgical instruments received in the container 3 for a doctor performing the operation to use is considerably decreased, so the doctor can easily and simply select the surgical instruments required for the operation and it is possible to prevent the operation from being delayed. Further, all of the surgical instruments for implant surgery do not need to be sterilized, so the costs for sterilization are reduced and the cost for an operation is reduced, thereby decreasing patient expenses. In addition, since the container 3 can be manufactured in a small size, the container can be conveniently carried. Further, users can select and purchase only k-th trays 11 corresponding to desired sizes in consideration of the frequency of use of surgical instruments having specific sizes.

Although various embodiments of the present invention by the applicant(s) were described above, the embodiments are just examples for achieving the spirit of the present invention and any modifications and changes should be construed as being included in the scope of the present invention as long as they can achieve the spirit of the present invention.

### REFERENCE NUMERALS

1: k-th tray unit

11: k-th tray

11a: hole of tray

111: bracket

113: handle

13: k-th set unit

131: implant unit

131a: scanning information implant

133b: spare implant

133: n-k surgical instrument

3: container

3a: hole of container

C: lid

S-11, S-13, S-15: right femoral trial

S-31, S-33, S-35: PS box cutting guide

S-51, S-53, S-55: chamfer cutting guide

S-71, S-73, S-75: left femoral trial

S-91, S-93, S-95: base plate trial

What is claimed is:

1. An orthopedic surgical set, comprising:
k-th set units where implants having different sizes selected on the basis of scanning information of a patient obtained by a medical scanner and selected in consideration of a range of errors, which may occur between the scanning information and actual information, to perform implant surgery on a specific portion, and different kinds of surgical instruments having sizes corresponding to the implants to be used in the implant surgery are classified by sizes together with corresponding implants; and
k-th tray units to which the k-th set units are fixed, respectively.

2. The orthopedic surgical set of claim 1, wherein the k-th set units each include:
an implant unit including one scanning information implant selected on the basis of the scanning information and one or more spare implants selected within the range of errors that may occur between the scanning information and the actual information; and
n-k-th surgical instruments used to perform the implant surgery and obtained by classifying different kinds of surgical instruments having sizes corresponding to the scanning implant and the spare implants by sizes corresponding to the implants, respectively.

3. The orthopedic surgical set of claim of claim 2, wherein the number of the k-tray unit is the same of the number of the implants included in the implant unit.

4. The orthopedic surgical set of claim 3, wherein the number of the spare implants is one to three.

5. The orthopedic surgical set of claim 3, wherein the number of the spare implants is two.

6. The orthopedic surgical set of claim 5, wherein the two spare implants are spare implants larger and smaller by one size than the scanning information implant.

7. The orthopedic surgical set of claim 3, wherein surgical instruments having one size or different kinds of implants are fixed in a spare space of any one of the k-trays.

8. The orthopedic surgical set of claim 1, wherein the implant unit includes any one kind of implant of implants that are used for artificial knee implant surgery.

9. The orthopedic surgical set of claim 1, wherein the implant unit includes any one kind of implant of implants that are used for artificial hip joint implant surgery.

10. The orthopedic surgical set of claim 1, wherein the implant unit includes any one kind of implant of implants that are used for artificial shoulder implant surgery. CM 11. The orthopedic surgical set of claim 1, wherein the k-th tray includes:
brackets protruding upward in a predetermined shape from a bottom thereof to fix surgical instruments and implants for an operation; and
handles formed in a predetermined shape at both side of a top of the k-th tray for a user to easily hold the k-th tray.

12. The orthopedic surgical set of claim 11, further comprising a rectangular parallelopiped container having an open top to receive one or more trays therein.

13. The orthopedic surgical set of claim 12, wherein a plurality of holes is formed through a front side or a predetermined side of the trays and the container so that high-temperature vapor for sterilization flows into the trays and the containers.
